(19) United States
(12) Patent Application Publication
(10) Pub. No.: US 2011/0189301 A1
(43) Pub. Date: Aug. 4, 2011

(54) METHOD FOR PREPARING CONTACT LENS-SHAPED AMNIOTIC DRESSING

(75) Inventors: Eun Kyung YANG, Cheongwon-gun (KR); Sam Hyun Jung, Cheongwon-gun (KR); Sung Po Kim, Cheongwon-gun (KR); Ji Hoon Joo, Cheongwon-gun (KR); Jong Won Lee, Cheongwon-gun (KR); Jong Myoung Hong, Cheongwon-gun (KR); Jae Chan Kim, Seoul (KR); Yeoun Sook Chun, Seoul (KR); Sung Chun Lee, Pyeongtaek-si (KR); Kwang III Baik, Hwaseong-si (KR)

(73) Assignee: BIOLAND LTD., Cheongwon-gun (KR)

(21) Appl. No.: 12/761,048

(22) Filed: Apr. 15, 2010

(30) Foreign Application Priority Data
Feb. 3, 2010 (KR) 10-2010-0009921

(51) Int. Cl. A61K 35/48; A61P 27/02; B29C 63/02

(52) U.S. Cl. 424/582; 156/245

(57) ABSTRACT

The present invention relates to a method for preparing a contact lens-shaped amniotic dressing and a contact lens-shaped amniotic dressing prepared therefrom for treating ocular surface diseases, which does not require the use of sutures or an adhesion material. The inventive contact lens-shaped amniotic dressing is capable of solving the problems associated with suturing an amniotic membrane, e.g., highly delicate surgical techniques of suturing, long surgery time, stitch abscess, granuloma formation, tissue necrosis, and discomfort of patients; and the problems associated with the use of a support, e.g., the elimination of the support by eye blinking, breaking of the support, and discomfort.

### FIELD OF THE INVENTION

The present invention relates to a method for preparing a contact lens-shaped amniotic dressing for treating ocular surface diseases, which does not require the use of sutures or an adhesion material.

### BACKGROUND OF THE INVENTION

An amniotic membrane, a membrane having the thickness of 0.02 to 0.5 mm that is positioned in the inner surface of the placenta, has a stacked structure of an epithelium, a basement membrane, a compact layer, a fibroblast layer, and a sponge layer. An amniotic membrane is avascular and does not express histocompatible antigens, and hence immunological rejection after its transplantation does not occur (Akle C A et al., Lancet, 2:1003-5 (1981); Dua H S et al., Br. J. Ophthalmol., 83:748-52 (1999)). Since the basement membrane of the amniotic membrane is composed of type IV collagen, laminin, and α6/β4 integrin, and an interstitium thereof is composed of type I collagen, type III collagen, hyaluronic acid, fibronectin, and α5/β1 integrin, an amniotic membrane can facilitate the division and migration of a cell.

In ophthalmology, the use of an amniotic membrane was first reported by De Rotth in 1940 for treatment of conjunctival tissue loss (De Rotth A., Arch. Ophthalmol., 23:525-5 (1940)). In 1993, Batle and Perdomo reintroduced the use of an amniotic membrane as a conjunctival substitute (Batle J F, Perdomo F J, Ophthalmol., 100:107 (1993)). Kim and Tseng then showed in 1995 that an amniotic membrane facilitated corneal surface reconstruction in rabbits after epithelial removal and limbal lamella keratectomy (Kim J C and Tseng S C, Cornea, 14:473-84 (1995)).

It has been reported that an amniotic membrane facilitates re-epithelialization of cornea and conjunctiva and inhibits inflammation, and hence its transplantation is effective in treating diseases accompanied by inflammation, e.g., persistent corneal epithelial defect and ulceration in clinical trials. An amniotic membrane acts as a basement membrane that is responsible for maintaining the corneal and conjunctival cell growth and keeping their characteristics, and therefore, it facilitates the migration of epithelial cells, reinforces the adhesion of basal epithelial cells, promotes epithelial differentiation, and prevents epithelial apoptosis to promote epithelial regeneration on wound healing. Further, an amniotic membrane has anti-inflammatory activity by adhering to an inflammatory cell to prevent its permeation. Based on such characteristics of an amniotic membrane, it has been used for treatment of various ocular surface diseases such as keratohelcosis, pterygium, symblepharon, and chemical burn. Also, it has been reported that an amniotic membrane contains various growth factors such as EGF, TGF-α, KGF, HGF, bFGF, TGF-β1, and TGF-β2 which are involved in wound healing, and these growth factors remain intact after eliminating the epithelial layer from an amniotic membrane (Noriko Koizumi et al., Current Eye Research, vol. 20, No. 3, 173-177 (2000)). Although an amniotic membrane is prepared as a biological transplantation material by an ordinary method that eliminates homogeneous as well as heterogeneous cells which may cause inflammatory and immunological responses, its effectiveness in wound healing is maintained due to the presence of intact growth factors. Thus, an amniotic membrane is an effective and safe biological transplantation material.

In order to treat corneal epithelial defect, required are a continuous supply of oxygen to the epithelial cell, maintaining the corneal epithelium sufficiently hydrated, and the removal of specific factors which cause the friction to an epithelial cell. If such conditions are met and the basement membrane has no defects, epithelial cells can proliferate. However, the conventional methods, except for the method using an amniotic membrane, do not satisfy said conditions. Since an amniotic membrane per se has sufficiently high moisture content, its transplantation does not disturb the moisture in the surface of cornea and prevents the eye from undergoing a mechanical friction damage caused by blinking. Further, an amniotic membrane shows a higher oxygen permeability than a therapeutic lens made of a synthetic polymer material, which is beneficial in the reconstruction of the corneal epithelial cell.

In order to apply an amniotic membrane to the treatment of ocular surface diseases, it is necessary to perform 10-0 nylon suturing requiring highly delicate surgical techniques and a long surgery time, which often causes side effects, such as stitch abscess, granuloma formation, tissue necrosis, and discomfort of patients when the suture is removed.

In order to solve said problems, a method of fastening an amniotic membrane to a support is disclosed in U.S. Pat. No. 7,494,802, and the product therefrom is commercialized as Prokera™ (BioTissue, USA). Prokera™, which is intended for use after ocular surgery, is made of a rigid polycarbonate and prepared by fixing an amniotic membrane on a body having on the upper part thereof a hole of 15-16 mm using an O-ring. However, the use of Prokera™ causes the following problems: i) the amniotic membrane does not cover the protruding peripheral region of the cornea; ii) the amniotic membrane may be dislocated by eye blinking; iii) a part of the amniotic membrane may be damaged by eye blinking before the epithelial cells become grown; and iv) the rigid material irritates the eye.

In order to solve such problems associated with the use of a support, the present inventors have developed a method for preparing a contact lens-shaped amniotic dressing, which make an amniotic membrane be fastened to the eye without using any support.

### SUMMARY OF THE INVENTION

Accordingly, it is an object of the present invention to provide a method for preparing a contact lens-shaped amniotic dressing.

It is another object of the present invention to provide a contact lens-shaped amniotic dressing prepared by the method according to the present invention.

In accordance with an aspect of the present invention, there is provided a method for preparing a contact lens-shaped amniotic dressing, which comprises:

(a) bringing an amniotic membrane in contact with a convex portion of a mold comprising a cylindrical portion and the convex portion at the upper part of the cylindrical portion such that the peripheral region of the membrane drapes around the cylindrical portion;

(b) placing a ring on the amniotic membrane covering the convex portion and fixing the ring on the side surface of the cylindrical portion;

(c) crosslinking the amniotic membrane; and

(d) cutting the amniotic membrane along the boundary between the convex portion and the cylindrical portion of the mold, to obtain a contact lens-shaped amniotic dressing.

In accordance with another aspect of the present invention, there is also provided a contact lens-shaped amniotic dressing prepared by the method according to the present invention.

The contact lens-shaped amniotic dressing prepared by the inventive method of the present invention is capable of solving the problems associated with suturing an amniotic membrane, e.g., highly delicate surgical techniques of suturing, long surgery time, stitch abscess, granuloma formation, tissue necrosis, and discomfort of patients; and the problems associated with the use of a support, e.g., the elimination of the support by eye blinking, breaking of the support, and discomfort, and thus, is useful for treating ocular surface diseases.

### BRIEF DESCRIPTION OF THE DRAWINGS

The above and other objects and features of the present invention will become apparent from the following description of the invention, when taken in conjunction with the accompanying drawings, which respectively show:

FIG. 1: a schematic cross-sectional view of the mold used for preparing the contact lens-shaped amniotic dressing according to the present invention;

FIG. 2: a perspective view of the O-ring used for preparing the contact lens-shaped amniotic dressing according to the present invention;

FIG. 3: a top perspective view of the complex of the amniotic membrane and the mold;

FIG. 4: the rotating and cutting operation to obtain the inventive contact lens-shaped amniotic dressing using a lathe;

FIG. 5: two photographs of the contact lens-shaped amniotic dressing prepared by the method according to the present invention;

FIG. 6: a top perspective view of the contact lens-shaped amniotic dressing having a hole in the middle of the dressing for securing vision;

FIG. 7: two photographs of the contact lens-shaped amniotic dressing having a hole in the middle of the dressing for securing vision;

FIG. 8: top perspective views of various contact lens-shaped amniotic dressings each having a number of holes in the middle of the dressing for securing vision;

FIG. 9: photographs of the inventive contact lens-shaped amniotic dressings each having holes of various shapes in the middle of the dressing for securing vision;

FIG. 10: result of hydrating amniotic dressings with or without crosslinking;

FIG. 11: results of transplanting various amniotic dressings prepared from bovine amniotic membrane using hexamethylene diisocyanate (HMDI) into animals.

FIG. 12: the degrees of evaluating corneal opacity after treating corneal epithelial burn-induced rabbits with human amniotic membrane, acellular bovine amniotic membrane, and a therapeutic lens as a control, respectively; and

FIG. 13: photographs of the inflammatory cell in parenchymatous cornea after treating corneal epithelial burn-induced rabbits with human amniotic membrane, acellular bovine amniotic membrane, and a therapeutic lens as a control, respectively.

### DETAILED DESCRIPTION OF THE INVENTION

Hereinafter, the present invention is described in detail.

Unless defined otherwise, technical and scientific terms used herein have the same meaning as commonly understood by one of ordinary skill in the art to which the invention belongs. Further, all documents mentioned herein are incorporated by reference in their entireties.

The term “amniotic dressing” as used herein refers to a medical device made of an amniotic membrane, and especially, indicates a medical device brought in contact with cornea to treat ocular surface diseases. Also, the term “contact lens-shaped amniotic dressing” as used herein refers to a dressing which has a similar hardness to a contact lens and can be used in an identical manner to typical contact lens by contacting with cornea without the use of sutures or an adhesion material. This term can be interchangeable with the term “amniotic contact lens.”

The present invention provides a method for preparing a contact lens-shaped amniotic dressing, which comprises: (a) bringing an amniotic membrane in contact with a convex portion of a mold comprising a cylindrical portion and the convex portion at the upper part of the cylindrical portion such that the peripheral region of the membrane drapes around the cylindrical portion; (b) placing a ring on the amniotic membrane covering the convex portion and fixing the ring on the side surface of the cylindrical portion; (c) crosslinking the amniotic membrane; and (d) cutting the amniotic membrane along the boundary between the convex portion and the cylindrical portion of the mold, to obtain a contact lens-shaped amniotic dressing.

Step (a) of the inventive method comprises bringing an amniotic membrane in contact with a convex portion of a mold comprising a cylindrical portion and the convex portion at the upper part of the cylindrical portion such that the peripheral region of the membrane drapes around the cylindrical portion.

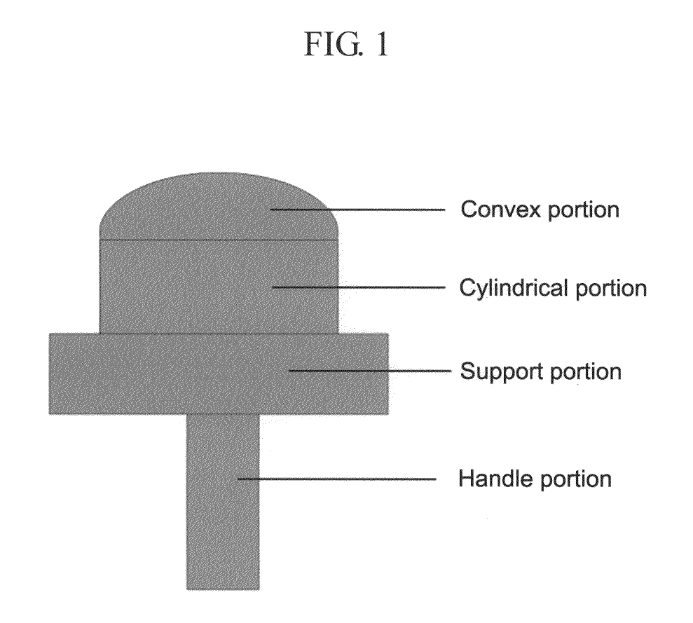

The mold comprises the cylindrical portion and the convex portion, and preferably has an appropriate diameter and base curve in order to shape into contact lens-shape. Specifically, the cylindrical portion preferably has a diameter of 8 to 22 mm and the convex portion preferably has a base curve of 6.0 to 10.0 mm. The mold may further comprise a support portion which supports the ring fixed on the lower part of the cylindrical portion for contacting and fastening an amniotic membrane, or a handle portion connected to the middle of the bottom surface of the support portion. As an example of the present invention, the mold illustrated by FIG. 1 can be used. The mold may be made of a material selected from the group consisting of an acrylic resin, an acetal resin, polytetrafluoroethylene (Teflon), stainless steel, and titanium, which, however, does not limit the scope of the present invention.

The amniotic membrane may be of a mammal origin, preferably a human or cattle origin. The amniotic membrane may be pre-washed or pretreated to remove its cells. The amniotic membrane can be washed by a method known in the art (see Kim J C and Tseng S C, Cornea, 14:473-84 (1995)) and pretreated with suitable solutions known in the art to eliminate its cells which may cause inflammatory and immunological responses. The amniotic membrane used in step (a) is stored under a cold condition until used.

The number of the amniotic membrane used may vary depending on the thickness, strength, and opacity of the amniotic membrane. Usually, one amniotic membrane is used, but, at least two overlapping layers of the amniotic membrane can be placed on the convex portion of the mold in order to obtain the desired strength of the amniotic dressing. Also, in order to solve the difficulty of securing vision caused by an opacity of an amniotic membrane itself, the method may further comprise forming a circular hole having a diameter of 1 to 7 mm in the middle of the amniotic membrane or forming at least two holes, preferably scores of holes, each having a diameter of 100 to 1,000 μm after step (a). In order to alleviate pain or a foreign body sensation due to an uneven surface caused by the holes, at least one amniotic membrane can be further placed on the existing membrane to form a multilayer of the amniotic membrane.

Step (b) of the present method comprises placing a ring on the amniotic membrane covering the convex portion and fixing the ring on the side surface of the cylindrical portion.

The ring (or “O-ring”) is used for contacting and fastening the amniotic membrane to the mold, and preferably manufactured to have an appropriate diameter in order to fit into the cylindrical portion of the mold. The ring may be made of a material selected from the group consisting of an acrylic resin, an acetal resin, polytetrafluoroethylene (Teflon), polyurethane, a rubber, a silicon rubber, and a perfluoroelastomer, which, however, does not limit the scope of the present invention. The amniotic membrane fastened to the mold may be dried with various drying procedures known in the art, for example, natural drying, freeze drying, and vacuum drying.

Step (c) of the inventive method comprises crosslinking the amniotic membrane.

When the dried amniotic membrane obtained in step (b) is hydrated, it cannot maintain a contact lens-shape and return to an original non-shaped soft membrane. Thus, in order to prevent it, the amniotic membrane should be subjected to a crosslinking procedure. The crosslinking can be conducted by a physical or chemical crosslinking treatment. The physical crosslinking treatment comprises UV irradiation and dehydrothermal treatment. UV irradiation treatment is preferably performed by irradiating the amniotic membrane with UV at wavelength of 254 to 305 nm for 10 to 240 minutes and dehydrothermal treatment may be performed by putting the amniotic membrane under the vacuum condition of at most 100 mTorr at the temperature of 90 to 105° C. for 60 minutes to 72 hours. The chemical crosslinking treatment may be performed by immersing the amniotic membrane in a solution selected from the group consisting of carbodiimide, 1-ethyl-3-(3-dimethylaminopropyl)carbodiimide (EDC or EDAC), glutaraldehyde, formaldehyde, hexamethylene diisocyanate (HMDI), dextran, and glucose.

Step (d) of the present method comprises cutting the amniotic membrane along the boundary between the convex portion and the cylindrical portion of the mold, to obtain a contact lens-shaped amniotic dressing.

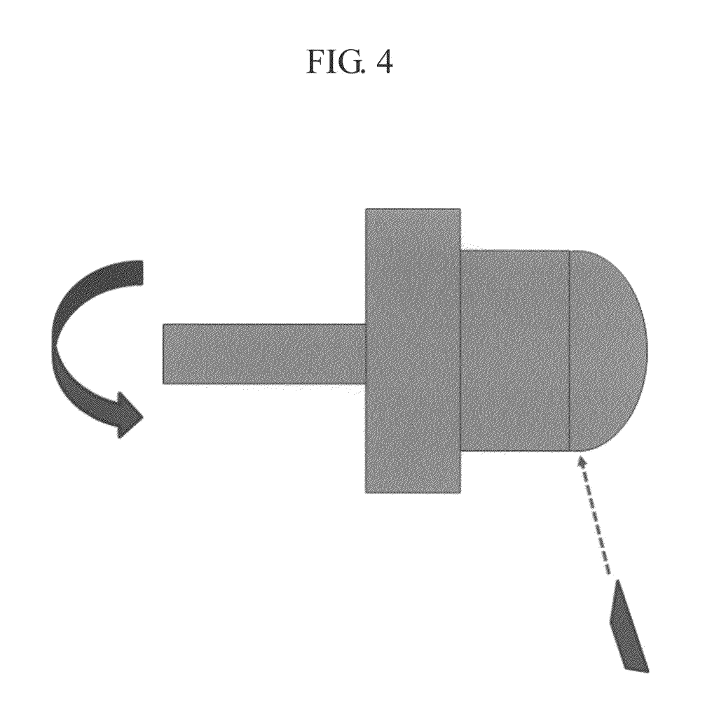

In order to obtain the contact lens-shaped amniotic dressing, the amniotic membrane is rotated and cut along the boundary between the convex portion and the cylindrical portion by using a lathe and laser (FIG. 4).

Further, the amniotic dressing obtained from the above step may be sterilized by gamma-ray irradiation or electron beam irradiation after step (d).

Also, the present invention provides a contact lens-shaped amniotic dressing prepared by the method according to the present invention for treating ocular surface diseases.

The following examples are intended to illustrate the present invention without limiting its scope.

### Example 1

### Preparation of an Amniotic Membrane

(1-1) Preparation of Human Amniotic Membrane

Human amniotic membrane was treated by the method disclosed by an article of Kim and Tseng (Kim J C and Tseng S C, Cornea, 14:473-84 (1995)). An amniotic membrane was isolated from the placenta of a pregnant who had taken Caesarean section. The pregnants had been prescreened for hepatitis B and C viruses, HIV and syphilis, and only those placentas of which the maternal bloods reveal negative serological results were used. The amniotic membrane thus obtained was washed sequentially several times with sterile saline solution and sodium hypochlorite, and washed repeatedly with purified water. The resulting membrane was stored under a cold condition (2˜8° C.) until used.

(1-2) Preparation of Bovine Amniotic Membrane

Bovine amniotic membrane was treated by the following procedure. The bovine placenta was obtained upon delivery, and the amniotic membrane was separated therefrom and washed several times with saline solution to remove debris and bloodstain. The resulting amniotic membrane was washed with sterile saline solution and sodium hypochlorite. The stromal layer of the amniotic membrane was removed therefrom using a scraper and washed repeatedly with sterile saline solution and purified water. The resulting membrane was stored under a cold condition (2˜8° C.) until used.

(1-3) Treatment of an Amniotic Membrane for Removing Inflammatory and Immunogenic Materials

Each of the amniotic membranes obtained in (1-1) and (1-2) was treated by the following procedure in order to prevent inflammatory and immunological responses. Human or bovine amniotic membrane was stored in sterile saline solution under a cold condition. 500 cm² of the separated amniotic membrane was immersed in 1 L of 95% ethanol and kept overnight in a cold storage to remove lipid components therefrom. The amniotic membrane was soaked in 1 L of purified water and washed three times for 10 minutes. And then, the stromal layer of the amniotic membrane was removed therefrom using a scraper. The resulting amniotic membrane was stored in 1 L of 70% ethanol under a cold condition over a day to inactivate viruses and immersed in 1 L of ethylenediamine tetraacetic acid (EDTA)/sodium chloride solution (pH 11) containing 0.2% of EDTA and 0.9% of sodium chloride. The resulting mixture was stirred for 1 hour at 150 rpm to eliminate soluble alkaline impurities. Thereafter, the resulting amniotic membrane was immersed in trypsin/EDTA/sodium chloride solution (pH 7.4) containing 0.05% of trypsin, 0.02% of EDTA, and 0.9% of sodium chloride and the resulting mixture was subjected to enzyme reactions for 1 hour at 37° C. The amniotic membrane thus obtained was soaked in 1 L of 70% ethanol and the resulting mixture was stirred for 1 hour at 150 rpm to remove residual lipids. The resulting amniotic membrane was subjected to sequential soaking and stirring with the alkaline solution (pH 11) for 1 hour at 150 rpm and an acid solution (pH 2) containing 0.2% of EDTA for 1 hour at 150 rpm, respectively, to make the amniotic membrane in a swollen state, followed by washing three times with 1 L portions of purified water for 30 minutes at 150 rpm. The resulting membrane was stored under a cold condition (2˜8° C.) until used.

### Example 2

### Preparation of a Mold and a Ring

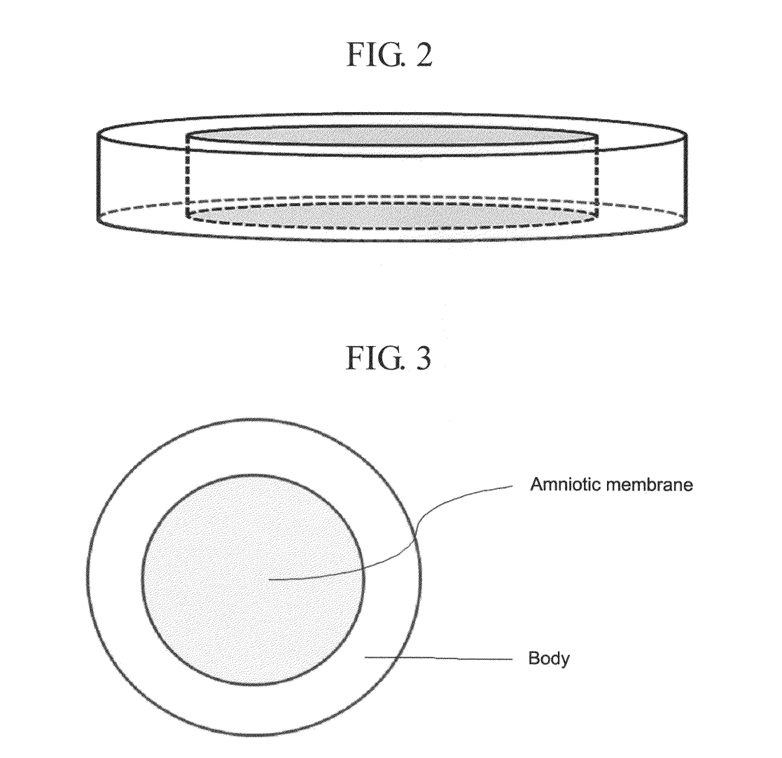

A mold for forming a contact lens-shaped amniotic membrane and a ring (or “O-ring”) for contacting an amniotic membrane with the mold were manufactured as shown in FIGS. 1 and 2. The mold was composed of a cylindrical portion having a diameter of 14 mm, a convex portion having a base curve of 8.4 to 8.8 mm, a support portion, and a handle portion. The support portion comprises a support which supports the ring used for contacting and fastening the amniotic membrane, and the handle portion has a cylindrical shape connected to the middle of the bottom surface of the support portion. The mold was made of an acetal resin. The O-ring of FIG. 2 was manufactured to fit the respective mold having a specific base curve. The O-ring was made of Teflon (14.1 mm internal diameter) or a perfluoroelastomer (12 mm internal diameter).

### Example 3

### Arrangement of the Amniotic Membrane

In order to make the amniotic membrane obtained in (1-1) to (1-3) have a contact lens-shape, the amniotic membrane was brought in contact with the contact lens-shaped mold obtained in Example 2, and then fixed on the mold with the O-ring.

Specifically, the amniotic membrane was cut to the size of the mold, and then was brought in contact with the convex portion of the mold (FIG. 1) with its epithelial layer facing downwards. In case of overlapping the amniotic membrane, the last amniotic membrane was overlapped so that its epithelial layer faced outside. The O-ring (FIG. 2) was placed thereafter on the amniotic membrane or overlapping amniotic membranes to completely cover the mold, followed by natural drying, freeze drying, or vacuum drying in a clean bench. A top perspective view of the dried amniotic membrane (including the mold) is shown in FIG. 3.

### Example 4

### Fixation of a Contact Lens-Shape of the Amniotic Membrane

(4-1) Shape Fixation Using a Physical Crosslinking Treatment

In case the dried amniotic membrane obtained in Example 3 is hydrated, it cannot maintain a contact lens-shape and return to an original plane and soft membrane. In order to prevent it, the complex of the mold, the O-ring, and the amniotic membrane obtained in Example 3 was subjected to the physical crosslinking procedure. Specifically, the complex was put into the vacuum of at most 100 mTorr at the temperature of 95° C., 100° C., or 105° C. for 48 to 72 hours.

(4-2) Shape Fixation Using a Chemical Crosslinking Treatment

To accomplish a more effective crosslinking than that achieved by the physical crosslinking treatment of Example (4-1), a chemical crosslinking treatment was conducted by using HMDI as a crosslinker. Specifically, the complex obtained in Example 3 was immersed in methanol solution containing 0.2% HMDI for 30 minutes to 12 hours, washed off the residual HMDI with methanol, and dried in a clean bench. Instead of said solution, 0.9% saline solution, ethanol (10˜100%), or methanol (10˜100%) containing 0.1˜3% HMDI, respectively, or 0.9% saline solution containing 0.1˜5% Tween 80 or Triton X-100 can be used.

### Example 5

### Cutting, Washing, and Sterilizing the Amniotic Membrane

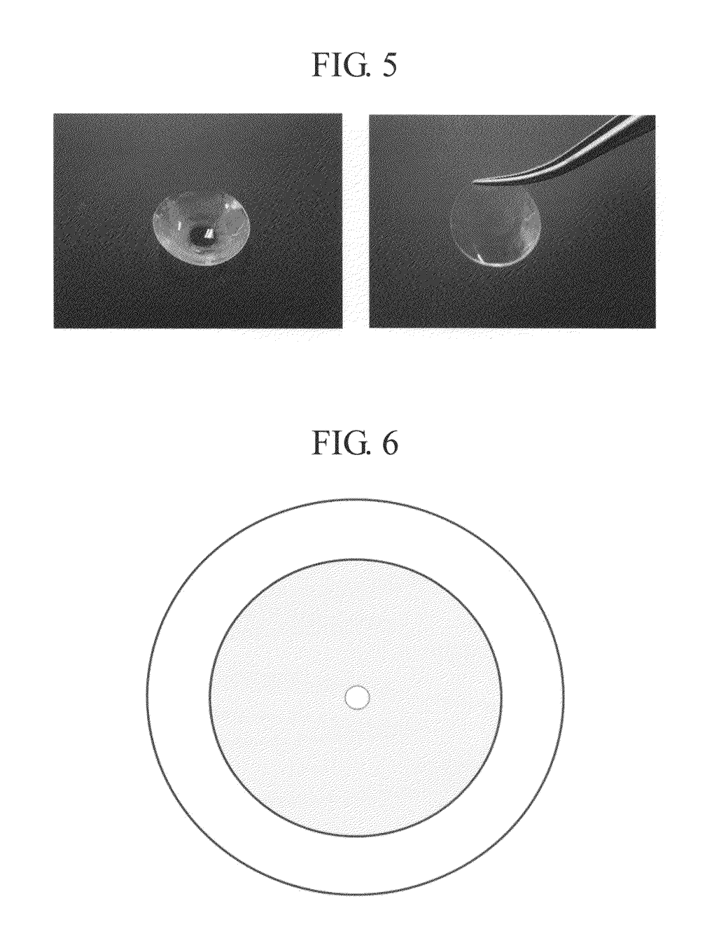

In order to isolate the contact lens-shaped amniotic membrane from the complex of the mold, the O-ring, and the amniotic membrane obtained in Example 4, the complex was combined with a rotator of a lathe to rotate with high speed and cut with a diamond cutting edge having a degree of precision of 1/100˜ 1/1000 mm as shown in FIG. 4. The resulting contact lens-shaped amniotic membrane was washed with Tween 80 solution or Triton X-100 solution (0.1˜5%), washed repeatedly with saline solution, and washed with purified water which had been filtered three times to completely remove the residual crosslinker. Thereafter, the amniotic membrane was dried, sealed, wrapped, and sterilized by gamma-ray irradiation at 10˜25 kGy or electron beam irradiation for the use as a dressing. The contact lens-shaped amniotic dressing thus obtained is shown in FIG. 5.

### Example 6

### Formation of a Hole for Securing Vision

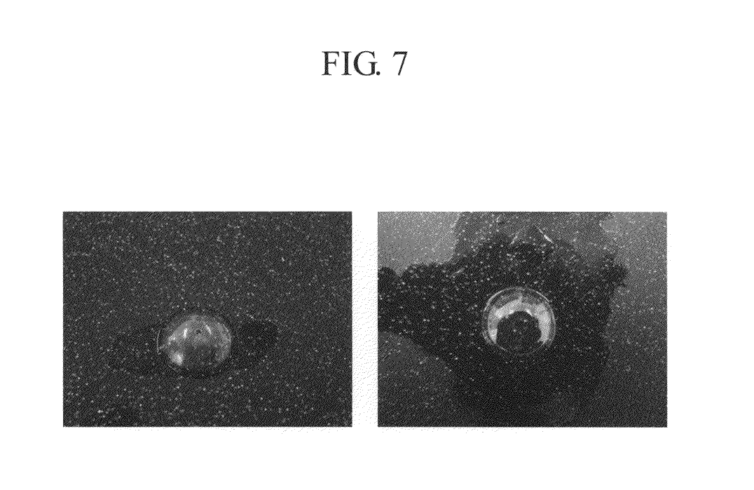

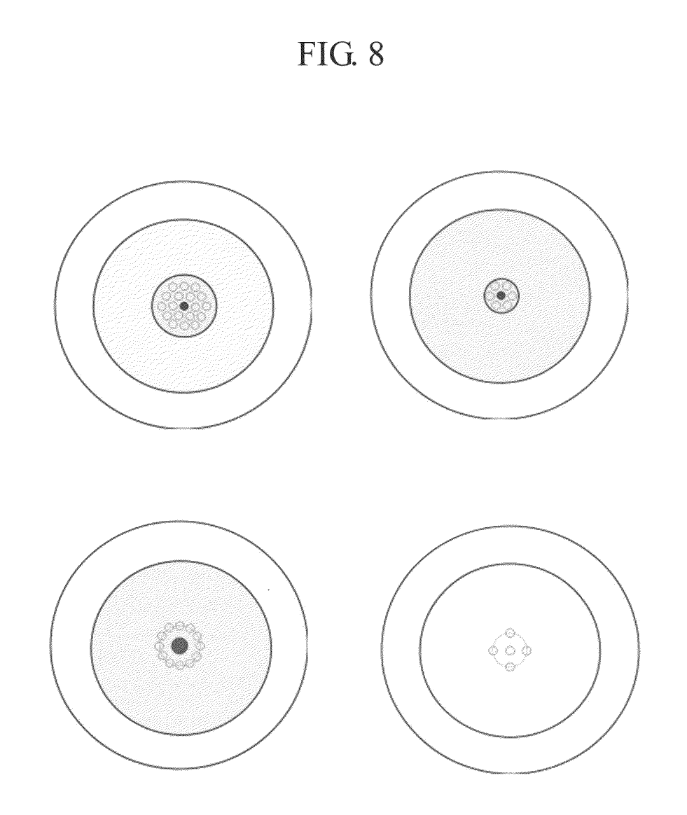

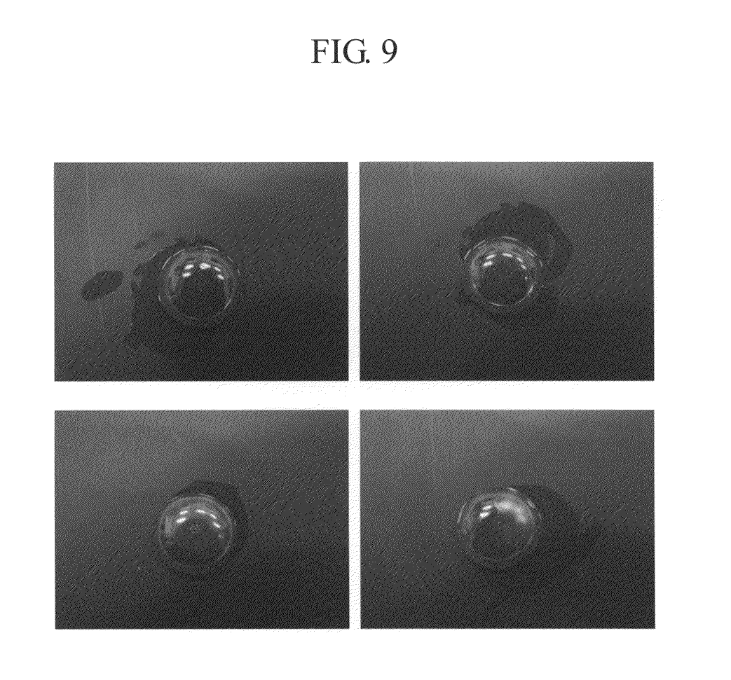

Although an amniotic membrane per se is transparent, it is difficult to see through overlapping layers of amniotic membranes because of their opacity. Thus, the amniotic membrane obtained in Example 3 was perforated with a lathe or CO₂ laser (GC111; EO Technics, South Korea) to make a hole having a diameter of 1 to 7 mm in the middle of it (FIG. 6 and FIG. 7). Further, instead of making one hole, a number of holes having a diameter of about 100 to 1,000 μm were formed in the middle of the amniotic membrane to secure vision (FIG. 8 and FIG. 9).

A green laser and UV laser can be also used instead of CO₂ laser.

Meanwhile, the perforated amniotic dressing may have an uneven surface at the perforated part, and therefore, the use of the amniotic dressing may irritate the inner surface of the eyelid when blinking to give a pain or a foreign body sensation to the eye. Thus, another amniotic membrane can be overlapped thereon after the perforation to alleviate the irritation.

### Experimental Example 1

### Crosslinking Effect of an Amniotic Dressing

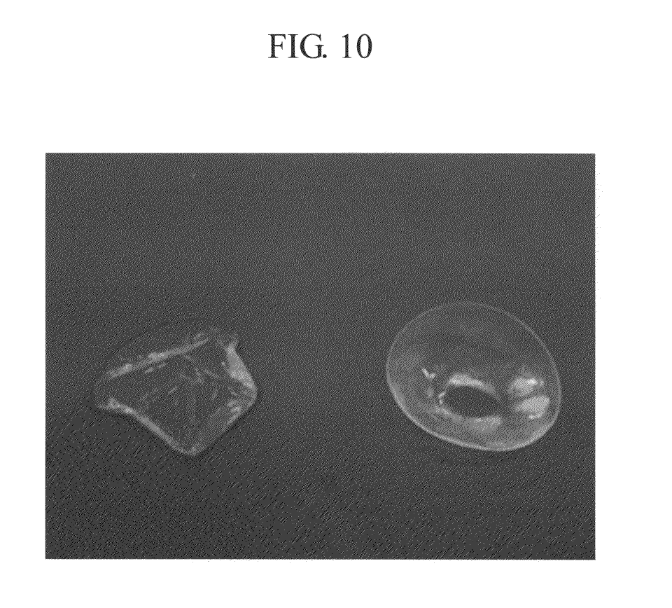

In order to investigate the shape fixing effect after the crosslinking treatment in Example 4, the amniotic membranes obtained in Example 3, which were cut after chemical crosslinking treatment (an experimental group) and cut without chemical crosslinking treatment (a control group), respectively, were subjected to the following procedure. The experimental and control groups were immersed in saline solution at room temperature for 30 minutes, and then, their shape was observed. As shown in FIG. 10, it was confirmed that while the control group lost its contact lens shape by hydration, the experimental group maintained its contact lens shape in spite of hydration. Therefore, it is found that the contact lens shape of the amniotic dressing can be effectively maintained by crosslinking.

### Experimental Example 2

### Quantitative Measurement of Growth Factor in the Amniotic Membrane

To determine the presence of growth factor in the amniotic membrane and the quantity thereof, the quantitative analysis on bFGF and KGF among various growth factors, which have a cross reactivity between human and bovine, was conducted by enzyme linked immunosorbent assay (ELISA; R&D system, USA). Human amniotic membrane, bovine amniotic membrane, and acellular bovine amniotic membrane obtained in Examples (1-1) and (1-2) were used as experimental samples. The samples were subjected to a freeze drying and ground to a powder in freezer mill (Spex, USA). 60 mg of the respective powdered samples were added to a tube containing 2 mL of phosphate buffered saline (PBS; pH 7.4) and growth factors were extracted five times with a one-minute interval by a homogenizer at 10,000 rpm under a cold condition. A supernatant was collected after centrifugation for 20 minutes at 13,000 rpm at 4° C. to measure the amount of growth factors by ELISA kit. Each quantity of the extracted growth factors was calculated based on the quantity of total proteins measured by Bradford assay. The contents of growth factors measured are shown in Table 1 below.

TABLE 1
Contents of growth factors in the amniotic membrane

| Subject | bFGF (pg/μg) | KGF (pg/μg) |
|---|---|---|
| Human amniotic membrane | 420.71 ± 23.23 | 70.13 ± 11.48 |
| (Example (1-1)) | | |
| Bovine amniotic membrane | 606.82 ± 28.93 | 29.13 ± 2.92 |
| (Example (1-2)) | | |
| Acellular bovine amniotic membrane | 41.67 ± 23.57 | 238.64 ± 40.18 |
| (Example (1-3)) | | |

As shown in Table 1, bFGF was present in the amount of 420.71±23.23 pg/μg in human amniotic membrane, 606.82±28.93 pg/μg in bovine amniotic membrane, and 41.67±23.57 pg/μg in acellular bovine amniotic membrane. KGF was present in the amount of 70.13±11.48 pg/μg in human amniotic membrane, 29.13±2.92 pg/μg in bovine amniotic membrane, and 238.64±40.18 pg/μg in acellular bovine amniotic membrane. That is, in spite of the difference in its relative amount, growth factors, bFGF and KGF, are contained in amniotic membrane regardless of the existence of a cell.

### Experimental Example 3

### Biocompatibility Test of the Amniotic Dressing—Subcutaneous Implantation to Guinea Pig

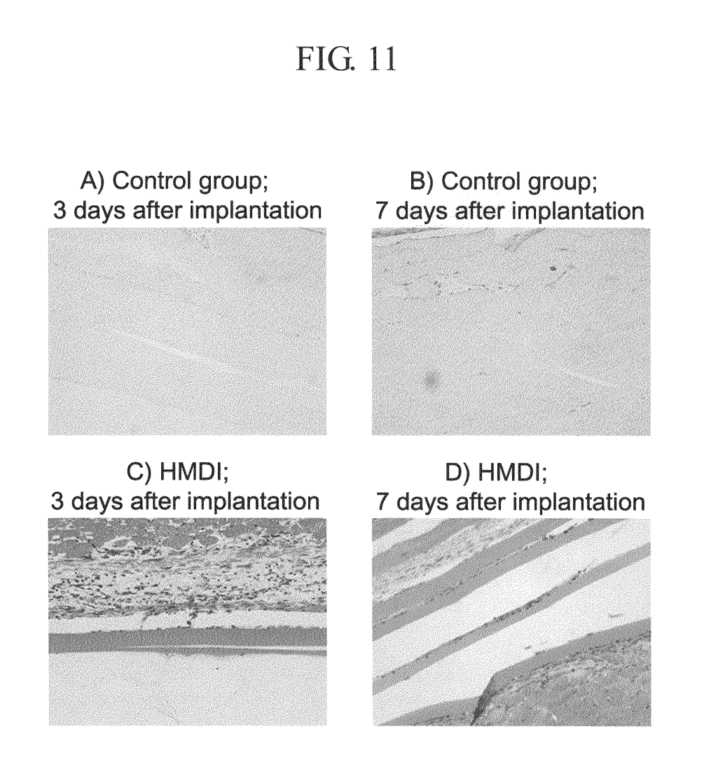

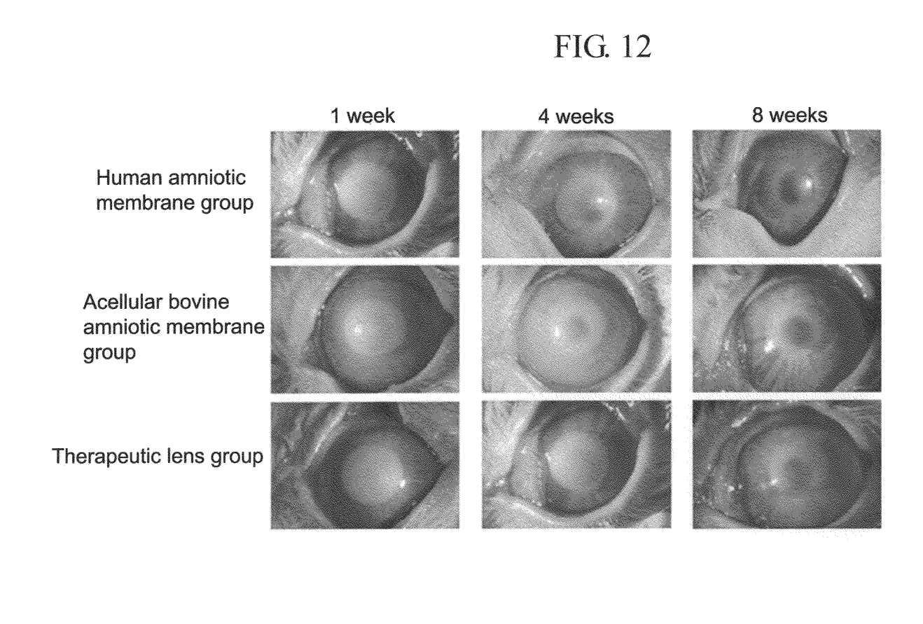

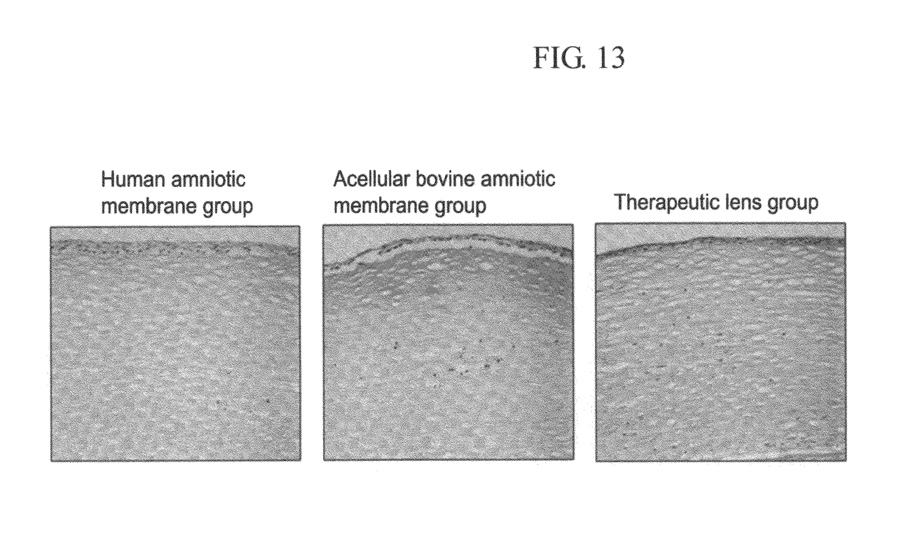

To investigate biocompatibility of the amniotic dressing according to the present invention, the contact lens-shaped amniotic dressings prepared by the chemical crosslinking treatment were used. The acellular bovine amniotic membrane obtained in Example (1-3) sterilized by gamma-ray irradiation was used as a control. The test was conducted by comparing the degree of inflammation and foreign body reaction in a guinea pig to which a subcutaneous implantation of each sample is applied. 3 or 7 days after the implantation, the applied tissue was taken from the each guinea pig, fixed with formalin, washed, and embedded with paraffin. The tissue thus obtained was cut to 5 μm of thickness and subjected to hematoxylin & eosin (H&E) staining. The stained tissue was then observed using an optical microscope. As a result, any inflammatory response and foreign body reaction were not observed in all the control and experimental group of day 3 and 7, and therefore, it is found that a contact lens-shaped amniotic dressing prepared by the chemical crosslinking treatment is biocompatible like bovine amniotic membrane itself (control) (FIG. 11).

### Experimental Example 4

### Effectiveness of Human Amniotic Membrane and Acellular Bovine Amniotic Membrane on Rabbit's Corneal Chemical Burn

To investigate the healing effect of human amniotic membrane and acellular bovine amniotic membrane obtained in Examples (1-1) and (1-3) on the corneal epithelium, these membranes as well as a therapeutic lens (Focus, CIBA Vision Corporation) were applied to a rabbit's cornea.

16 New Zealand White rabbits (32 eyes) were used. In order to induce a chemical burn to the corneal epithelium, a circular filter paper sheet of 6.5 mm in diameter soaked with 0.1 N of NaOH was brought in contact with rabbit's cornea for 25 seconds. After removal of the filter paper sheet, the rabbit's eyes were washed with saline solution. 1 rabbit (2 eyes) was allowed to stand without any treatment, and remaining 15 rabbits (30 eyes) are divided into 3 groups where the rabbit's eyes of each group (5 rabbits, 10 eyes) were applied with human amniotic membrane, acellular bovine amniotic membrane, and therapeutic lens, respectively, followed by carrying out a tarsorrhaphy with a 6-0 black silk suture. After the surgery, cravit (levofloxacin) and maxitrol were dropped into the eyes 4 times daily, respectively. The healing degree of the corneal epithelium was examined daily with a slit lamp and the respective amniotic membranes were applied until the chemical burn was healed. The clinical condition was evaluated by measuring i) the healing time of the corneal epithelium, ii) the degree of corneal edema, and iii) the degree of corneal opacity at 1 week, 4 weeks and 8 weeks after the surgery, respectively. The degree of corneal edema was evaluated by measuring the central corneal thickness with a pachymeter and the degree of corneal opacity was graded from 0 (transparent) to 4 (totally opaque) according to Fantes' classification. Further, at 1 week after the surgery, the corneal tissue was taken and stained with H&E to observe iv) the number of inflammatory cells in the cornea at continuous 5 microscopic views (×200). The results are shown in Tables 2 to 5.

TABLE 2
The healing time of the corneal epithelium

| | Groups | | |
|---|---|---|---|
| | Human amniotic | Acellular bovine | Therapeutic |
| | membrane | amniotic membrane | lens |
| The healing time | 3.2 ± 0.42 | 3.3 ± 0.48 | 3.3 ± 0.48 |
| (day) | | | |

There was no significant difference in the healing time of the corneal epithelium among the test groups.

TABLE 3
Corneal thickness

| Groups | 1 week | 4 weeks | 8 weeks |
|---|---|---|---|
| Human | 754.4 ± 47.6 μm | 691 ± 57.3 μm | 636.9 ± 45.2 μm |
| amniotic | | | |
| membrane | | | |
| Acellular | 775 ± 44.4 μm | 724.6 ± 45.4 μm | 659.6 ± 46.3 μm |
| bovine | | | |
| amniotic | | | |
| membrane | | | |
| Therapeutic | 814.2 ± 56.9 μm | 773.1 ± 55.0 μm | 716.1 ± 52.5 μm |
| lens | | | |

The corneal edema can be measured by observing corneal thickness and there was a significant improvement 4 weeks later in the corneal edema of human amniotic membrane group and acellular bovine amniotic membrane group.

TABLE 4
Corneal opacity

| Groups | 1 week | 4 weeks | 8 weeks |
|---|---|---|---|
| Human amniotic | 3.7 ± 0.48 | 3.0 ± 0.47 | 2.5 ± 0.53 |
| membrane | | | |
| Acellular bovine | 3.8 ± 0.42 | 3.2 ± 0.32 | 2.8 ± 0.52 |
| amniotic membrane | | | |
| Therapeutic lens | 3.9 ± 0.32 | 3.6 ± 0.52 | 3.1 ± 0.32 |

As shown in Table 4 and FIG. 10, there was a significant improvement 4 weeks later in the corneal opacity of human amniotic membrane group and acellular bovine amniotic membrane group.

TABLE 5
The number of inflammatory cells in parenchymatous cornea

| | Groups | | |
|---|---|---|---|
| | Human amniotic | Acellular bovine | Therapeutic |
| | membrane | amniotic membrane | lens |
| The number of | 9.2 ± 2.7 | 9.4 ± 3.1 | 15.3 ± 5.3 |
| inflammatory | | | |
| cells | | | |

As shown in Table 5 and FIG. 11, the number of inflammatory cells in parenchymatous cornea of human amniotic membrane group and acellular bovine membrane group decreased significantly compared to that of therapeutic lens group.

As discussed above, the healing time and healing degree of the corneal epithelium were evaluated by comparing cell-containing human amniotic membrane group and acellular bovine amniotic membrane group with therapeutic lens group, and it is found that there is no significant difference in the healing time among the test groups, but human amniotic membrane group and acellular bovine amniotic membrane group have anti-inflammatory activity to inhibit corneal edema and corneal opacity.

What is claimed is:

1. A method for preparing a contact lens-shaped amniotic dressing, which comprises:
(a) bringing an amniotic membrane in contact with a convex portion of a mold comprising a cylindrical portion and the convex portion at the upper part of the cylindrical portion such that the peripheral region of the membrane drapes around the cylindrical portion;
(b) placing a ring on the amniotic membrane covering the convex portion and fixing the ring on the side surface of the cylindrical portion;
(c) crosslinking the amniotic membrane; and
(d) cutting the amniotic membrane along the boundary between the convex portion and the cylindrical portion of the mold, to obtain a contact lens-shaped amniotic dressing.

2. The method of claim 1, wherein a diameter of the cylindrical portion is 8 to 22 mm and a base curve of the convex portion is 6.0 to 10.0 mm.

3. The method of claim 1, wherein the mold is made of a material selected from the group consisting of an acrylic resin, an acetal resin, polytetrafluoroethylene (Teflon), stainless steel, and titanium.

4. The method of claim 1, wherein the mold further comprises a support portion which supports the ring placed on the side surface of the cylindrical portion.

5. The method of claim 1, wherein the amniotic membrane is of a human or cattle origin.

6. The method of claim 1, wherein the amniotic membrane is pre-washed or pretreated to remove its cells.

7. The method of claim 1, wherein at least two overlapping layers of the amniotic membrane are placed on the convex portion of the mold.

8. The method of claim 1, wherein the ring is made of a material selected from the group consisting of an acrylic resin, an acetal resin, polytetrafluoroethylene (Teflon), polyurethane, a rubber, a silicon rubber, and a perfluoroelastomer.

9. The method of claim 1, wherein the crosslinking is performed by UV irradiation or dehydrothermal treatment.

10. The method of claim 1, wherein the crosslinking is performed by immersing the amniotic membrane in a solution selected from the group consisting of carbodiimide, 1-ethyl-3-(3-dimethylaminopropyl)carbodiimide (EDC or EDAC), glutaraldehyde, formaldehyde, hexamethylene diisocyanate (HMDI), dextran, and glucose.

11. The method of claim 1, wherein the cutting is conducted by using a lathe and laser.

12. The method of claim 1, which further comprises sterilizing the amniotic dressing by gamma-ray irradiation after step (d).

13. The method of claim 1, which further comprises forming a circular hole having a diameter of 1 to 7 mm in the middle of the amniotic membrane or forming at least two holes each having a diameter of 100 to 1,000 μm after step (a).

14. The method of claim 13, which further comprises placing at least one amniotic membrane to overlap the treated membrane obtained after forming the hole or holes.

15. A contact lens-shaped amniotic dressing prepared by the method according to claim 1 for treating ocular surface diseases.
